(19) United States
(12) Patent Application Publication
(10) Pub. No.: US 2006/0240538 A1
(43) Pub. Date: Oct. 26, 2006

(54) Cell separation method using alumina

(76) Inventors: Hee-kyun Lim, Suwon-si (KR); Chang-eun Yoo, Seoul (KR); Kyu-youn Hwang, Incheon-si (KR); Hun-joo Lee, Seoul (KR); Joon-ho Kim, Seongnam-si (KR)

Correspondence Address: CANTOR COLBURN, LLP, 55 GRIFFIN ROAD SOUTH, BLOOMFIELD, CT, 06002, US

(21) Appl. No.: 11/338,981

(22) Filed: Jan. 25, 2006

(30) Foreign Application Priority Data
Jan. 25, 2005 (KR) 10-2005-0006577

(51) Int. Cl. C12N 7/02

(52) U.S. Cl. 435/239

(57) ABSTRACT

A method of separating cells or viruses using alumina is provided. The method includes: contacting a solution containing cells or viruses with alumina; and washing the alumina having cells or viruses bound thereto. According to the method, the separation efficiency of cells or viruses can be increased, the detection limit for cells or viruses can be improved, and a sample can be rapidly prepared.

### CROSS-REFERENCE TO RELATED PATENT APPLICATION

This application claims the benefit of Korean Patent Application No. 10-2005-0006577, filed on Jan. 25, 2005, in the Korean Intellectual Property Office, the disclosure of which is incorporated herein in its entirety by reference.

### BACKGROUND OF THE INVENTION

1. Field of the Invention

The present invention relates to a cell separation method using alumina.

2. Description of the Related Art

Conventional methods of purifying bacterial and viral nucleic acids have significant faults. Conventional protocols for purifying bacterial and mammalian viruses from host cells or growth media generally contain three steps. First, viruses must be liberated from the host cells. Second, the virus must be concentrated prior to actual purification. Third, the concentrated virus must be purified from extraneous materials.

These conventional methods of isolating viruses from biological fluids, aqueous suspensions or solutions comprising biological fluids, require either exceedingly long times or expensive equipment for the centrifugation; and further require use of toxic chemicals.

One approach to improving virus-isolating techniques has been to selectively adsorb viruses onto a solid material. An ideal adsorbent would selectively adsorb virus under certain conditions from extraneous materials in liquid suspensions, and desorb viable viruses under different condition to permit physical separation of viral particles.

Various synthetic polymeric materials have been employed in this approach. Synthetic polymeric materials which are water insoluble have also been employed in attempts to adsorb viruses. Most of these however have been pH insensitive, so that desorption of viruses would not occur upon change of pH. Also, certain investigators have employed synthetic water-insoluble polymeric materials to adsorb viruses at acidic pH and to desorb them at elevated pH. However, these materials have generally been used to treat only very high volumes of water intended for drinking.

U.S. Pat. No. 5,658,779 describes a method of adsorbing viruses from a fluid composition. In the method, viruses or viral nucleic acids are removed, purified or recovered using a polycarboxylic acid polymer. There is no description regarding use of alumina in separation of cells or viruses.

When nucleic acids are isolated from bacteria or viruses, if cells or viruses have a very low initial concentration, they should be concentrated. In particular, a small chip requires a small volume of sample. Therefore, studies on cell concentration are further required.

The inventors of the present invention studied a separation method of cells or viruses based on the conventional technologies and found that the separation efficiency of cells or viruses is increased by using alumina prepared using a method according to an embodiment of the present invention.

### SUMMARY OF THE INVENTION

The present invention provides a method of separating cells using alumina to increase the separation efficiency of cells or viruses.

According to an aspect of the present invention, there is provided a method of separating cells or viruses using alumina according to an embodiment of the present invention includes: contacting a solution containing cells or viruses with alumina; and washing the alumina having cells or viruses bound thereto.

### BRIEF DESCRIPTION OF THE DRAWINGS

The above and other features and advantages of the present invention will become more apparent by describing in detail exemplary embodiments thereof with reference to the attached drawings in which:

FIG. 1 is a schematic view of a method of separating cells using alumina according to an embodiment of the present invention, followed by separating nucleic acids from the separated cells;

FIG. 2 illustrates XPS analysis results of alumina using a glass plate, alumina prepared from aluminium ethoxide and alumina prepared from aluminium chloride;

FIG. 3 illustrates microscopic images of stained Bacillus and E. coli cells which are bound to an alumina substrate;

FIG. 4 illustrates microscopic images showing the binding ability of Bacillus cells with respect to pH and the type of substrate;

FIG. 5 illustrates microscopic images showing the binding ability of E. coli cells with respect to pH and the type of substrate;

FIG. 6 illustrates microscopic images showing the binding ability of E. coli cells with respect to binding time;

FIG. 7 illustrates the fluorescence intensity of Cy3-labeled IgG bound to a carboxyl substrate, an amine substrate and an alumina substrate of the present invention with respect to pH;

FIG. 8 illustrates microscopic images showing the binding ability of E. coli cells bound to an alumina substrate of the present invention with respect to the presence or absence of IgG;

FIG. 9 illustrates the fluorescence intensity of DNA bound to an alumina substrate of the present invention with respect to the type of buffer;

FIG. 10 is a graph illustrating the crossing point (Cp) of DNA separated using a method according to an embodiment of the present invention; and

FIG. 11 is a microscopic image showing the binding ability of E. coli bound to an alumina substrate of the present invention.

### DETAILED DESCRIPTION OF THE INVENTION

A method of separating cells or viruses using alumina according to an embodiment of the present invention includes: contacting a solution containing cells or viruses with alumina; and washing the alumina having cells or viruses bound thereto.

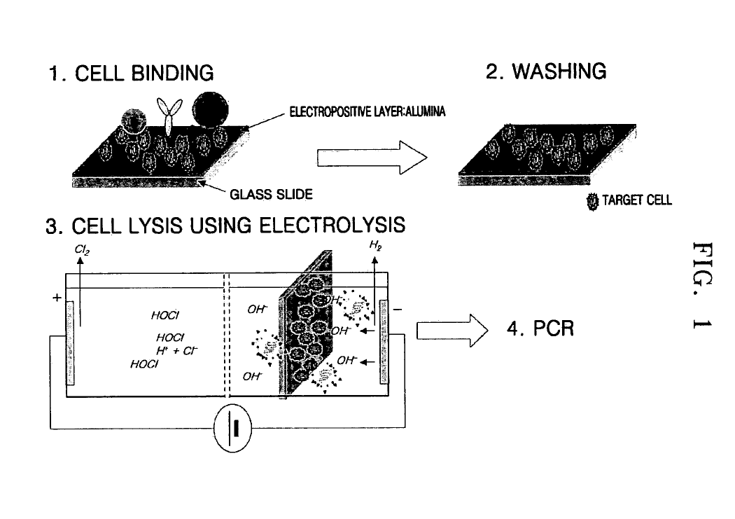

FIG. 1 is a schematic diagram of a method of separating cells using alumina according to an embodiment of the present invention, followed by separating nucleic acids from the separated cells. When a solution containing cells or viruses is contacted with alumina of the present invention, the cells or viruses bind to alumina due to electrostatic interaction, hydrophobic interaction thereof with alumina. When the alumina having cells or viruses bound thereto is washed with a washing solution such as a phosphate buffer, materials that do not bind to the alumina are removed and cells or viruses that are target materials primarily bind to the alumina. Thus, cells or viruses can be selectively separated, and thus the effects of concentrating cells or viruses can also be obtained.

The alumina used in the method is prepared using a general sol-gel method illustrated below.

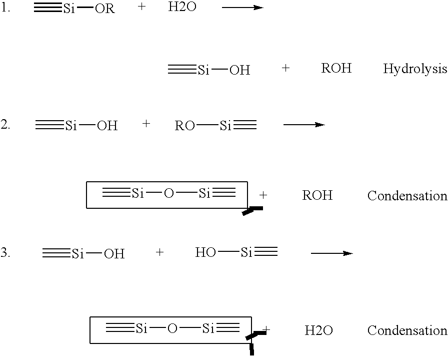

In the present invention, alumina is prepared using aluminium ethoxide or aluminium chloride as a precursor according to the following reaction.

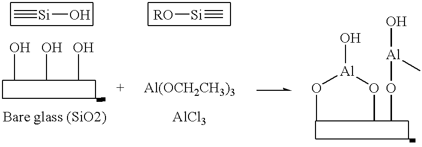

A medium in which the precursor is dissolved includes any appropriate organic solvent or a mixture of organic solvents. The medium may be preferably a polar organic solvent, such as alcohol, ketone, ester or ether. Alcohol is more preferable. Examples of appropriate alcohol include lower alcohols such as methanol, ethanol, isopropanol and butanol. Ethanol, isopropanol or a combination thereof is preferable.

The amount of the precursor dissolved in the medium depends on several factors such as the surface area of particles to be coated, the coordination number of oxide to be formed, etc.

Next, the precursor solution is treated such that the dissolved aluminium can be ionized or hydrated. This is achieved by hydrolyzing the precursor. The hydrolysis of precursor can be carried out by any method known in the art, for example, by contacting the precursor solution with water or aqueous base. For example, water or base is added to the precursor solution, and then the mixture is heated, and preferably strongly stirred.

In the hydrolysis, water is preferably used in an excess amount with respect to aluminium ethoxide. Thus, the mole ratio of water to precursor is at least about 10:1, and preferably at least about 100:1, and more preferably about 100:1 to 300:1.

The hydrolysis can be accelerated by heating the precursor solution. For example, the precursor solution can be heated to a temperature of about 40-100° C., preferably about 50-85° C. In a specific embodiment, the solution is heated to a solvent reflux temperature. The heating is carried out until the hydrolysis sufficiently occurs, preferably is completed. Since the hydrolysis rate is proportional to temperature, the heating time depends on temperature. As temperature increases, the heating time decreases. The solution can be heated for about 0.1 hour or longer, for example about 1-72 hours. The heating time is preferably about 10-30 hours, and more preferably about 20-24 hours.

The pH of the solution containing aluminium ethoxide has an important effect on the quality of a finally obtained coating. To obtain a thin, smooth and continuous coating, heterogeneous nucleation of aluminium ethoxide is preferable. It was confirmed that such a nucleation can be accomplished by adjusting the pH of a hydroxide solution to, for example, about 4.0-10.0, and preferably about 5.0-8.0, and more preferably neutral pH, for example, 7.5.

A method of separating nucleic acids from cells or viruses using alumina according to another embodiment of the present invention includes: contacting a solution containing cells or viruses with alumina; washing the alumina having cells or viruses bound thereto; and lysing the cells or viruses bound to the alumina.

To separate nucleic acids from cells or viruses separated by the above method, lysis of cells or viruses is required. For implementation of a Lab-On-a-Chip (LOC), the separation of nucleic acids following the separation of cells is required. According to the method of the present embodiment, cells or viruses can be rapidly concentrated or separated and the separated cells or viruses which are bound to alumina can be lysed by various cell lysis methods, to release nucleic acids. The released nucleic acids should exist in the solution without adsorbing to alumina in order to be used in a subsequent step. The alumina of the present invention does not adsorb nucleic acids released from the lysed cells or viruses, and thus is suitable for implementation of LOC.

In an embodiment of the present invention, the lysis of cells or viruses can be carried out by electrolysis. Cell lysis is generally carried out using mechanical, chemical, thermal, electrical, ultrasonic and microwave methods (Michael T. Taylor et al., Anal. Chem., 73, 492-496 (2001)). Preferably, cell lysis is carried out by electrolysis. Electrolysis can simply lyse cells or viruses by simply applying electric field without using chemicals. Also, since chemicals which can act as PCR inhibitors are not used, nucleic acids can be easily amplified in a subsequent amplification step. Further, electrodes can be easily patterned in the application to a microsystem.

In an embodiment of the present invention, the alumina may be in the form of particle, membrane or substrate. Alumina means aluminium oxide (Al₂O₃) and has various forms such as crystal, powder, membrane, plate and bead. Preferably, the alumina is in a substrate form.

In an embodiment of the present invention, the alumina may be prepared from aluminium ethoxide or aluminium chloride. Such an alumina can have various characteristics and is most suitable for the purpose of the present invention. The alumina is identified using X-ray photoelectron spectroscopy (XPS). XPS is a device for chemically analyzing a sample by irradiating gentle X-ray of about 1 KeV to a sample and detecting electrons emitted through photoemission.

The basic principle is as follows. When light emitted from a certain light source passes through a sample, the following photoemission occurs.
hν=Ek+EB−Φ(ν: frequency, Ek: kinetic energy, EB: binding energy, Φ: work function)

When the energy of light source and the work function of a sample are known, the energy of detected electrons is found out, and then the binding energy of the sample can be found out. Every material has an intrinsic electron state applied to each orbital and detected only at a particular binding energy. That is, when a binding energy spectrum of a sample is obtained using XPS, it can be seen a material constituting the sample.

In an embodiment of the present invention, the alumina may be prepared by sintering aluminium ethoxide or aluminium chloride at a temperature of 80-200° C. Out of the above temperature range, the separation effect of cells or viruses cannot be obtained.

In an embodiment of the present invention, the cells or viruses may be contacted with the alumina at pH 3-4.5. In the contact of cells or viruses with alumina, the pH is very important. As described in the following Examples, when a pH of a solution containing cells or viruses is out of the above range, cells or viruses hardly bind to alumina.

In the method of the present invention, the separated nucleic acid can be used to directly perform a nucleic acid amplification reaction, for example, a polymerase chain reaction (PCR), a ligase mediated chain reaction (LCR), a strand displacement amplification (SDA), a nucleic acid sequence based amplification (NASBA), a rolling circle amplification (RCA). Preferably, a PCR is performed. For the implementation of LOC, it is required that cells or viruses are separated, and an nucleic acid is separated, and then the separated nucleic acid is directly subjected to a PCR. The nucleic acid separated using the method of the present invention can be used to directly perform a PCR. As can be seen from the following examples, even when nucleic acids separated from cells or viruses are directly subjected to a PCR without purification, the PCR is effectively performed.

The present invention will now be described in greater detail with reference to the following examples. The following examples are for illustrative purposes only and are not intended to limit the scope of the invention.

### EXAMPLE 1

### Manufacturing and Analysis of Alnumina Substrate

Aluminium ethoxide or aluminium chloride was used as an alumina precursor to manufacture an aluminium substrate of the present invention. The detailed manufacturing method was as follows.

1. Washing of Glass Plate

Glasses were immersed in a piranah solution for at 2 hours or more. Then, the glasses were spin dried one by one.

2. Preparation of Aluminium Ethoxide Solution

120 mL of ethanol and a 100 mM aluminium ethoxide or aluminium chloride were combined and mixed for 1 hour. 500 μl of deionized water was added to the mixture and mixed for 1 hour.

3. Immersion of Glass

The glass prepared above 1 was immersed in the solution prepared above 2 for 2 hours.

4. Washing of Glass

The glass was twice washed with 800 mL of ethanol for each 10 minutes and washed with 800 mL of water for 5 minutes to hydrolyse an ethoxy group. Then, the glass was washed with 800 mL of ethanol for 5 minutes, and then dried in vacuum.

5. Sintering

An incubation was performed at 180° C. for 1 hour.

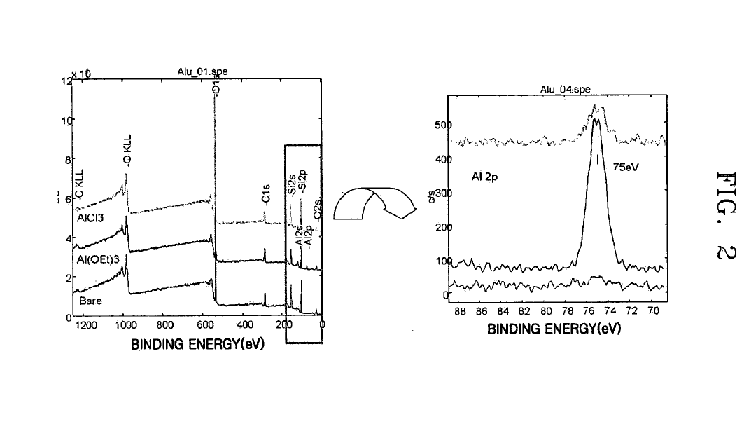

An XPS analysis for the prepared alumina was conducted. FIG. 2 illustrates the XPS analysis results for a glass plate, alumina prepared from aluminium ethoxide and alumina prepared from aluminium chloride. Referring to FIG. 2, when the aluminium ethoxide is used, the best result is obtained. When the alumina precursor includes an ethoxy group and hydrolysis is performed, polymerization is possible, thereby efficiently depositing an aluminium oxide film.

### EXAMPLE 2

### Separation of Bacterial Cells Using Alumina Substrate of the Present Invention

To find out whether bacterial cells can efficiently be separated by the method of the present invention, Bacillus subtilis as a gram positive cell and Escherichia coli (E. coli) as a gram negative cell were used. 60 μl of Bacillus subtilis cells and 60 μl of E coli cells suspended in a 0.1 M phosphate buffer (pH 4) and a 0.1 M phosphate buffer (pH 7), respectively, were allowed to bind to the alumina substrate prepared in Example 1. Then, cells bound to the alumina were twice washed with 30 mL of a 0.1 M phosphate buffer (pH 4) and 30 mL of a 0.1 M phosphate buffer (pH 7) for 5 minutes. After washing, the cells bound to the substrate were immobilized to perform a gram stain. The immobilization was achieved by two or three times heating the region to which cells were bound with an alcohol lamp for 1 or 2 seconds. Then, a gram stain was performed. First, a crystal violet solution was sufficiently applied to the region to which cells were bound. After 1 minute, the region was washed with flowing water. Then, a gram iodine solution, a gram decolorizer, and a gram safranin solution were treated in the same manner to complete the gram stain. After the gram stain, the alumina substrate was air dried at room temperature.

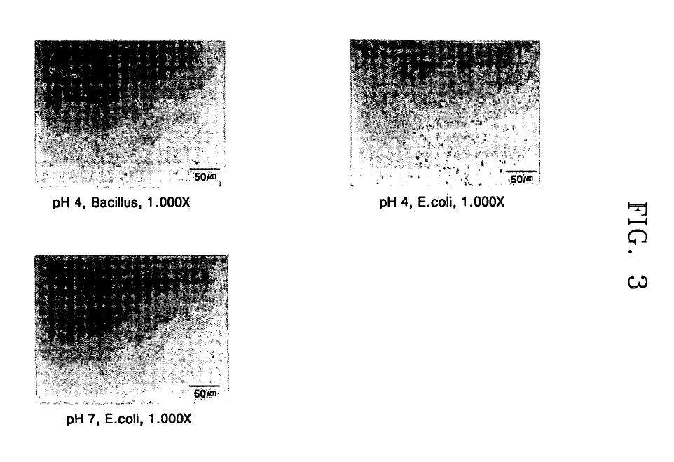

FIG. 3 is microscopic images showing the stain results of Bacillus subtilis and E. coli cells bound to the alumina substrate. Referring to FIG. 3, both of Bacillus subtilis and E. coli cells satisfactorily bind to the alumina substrate at pH 4, while both of Bacillus subtilis (not shown) and E. coli cells do not bind to the alumina substrate at pH 7.

### EXAMPLE 3

### Binding Ability of Bacillus subtilis Cells with Respect to pH and Type of Substrate

To investigate the binding ability of cells with respect to pH and the type of substrate, Bacillus subtilis cells and 0.1 M phosphate buffers of pH 4-10 were used. The alumina substrate of the present invention and a carboxyl substrate were used, in which the carboxyl substrate was prepared by immersing a bare glass in a 100 mM 3-triethoxysilylsuccinic anhydride in ethanol for 1 hour, twice washing with 800 mL of ethanol, immersing in 800 mL of water for 30 minutes, twice washing with 800 mL of ethanol and drying in vacuum. The separation and staining procedures of cells were performed in the same manner as Example 2, except that the binding time was 10 minutes instead of 30 minutes.

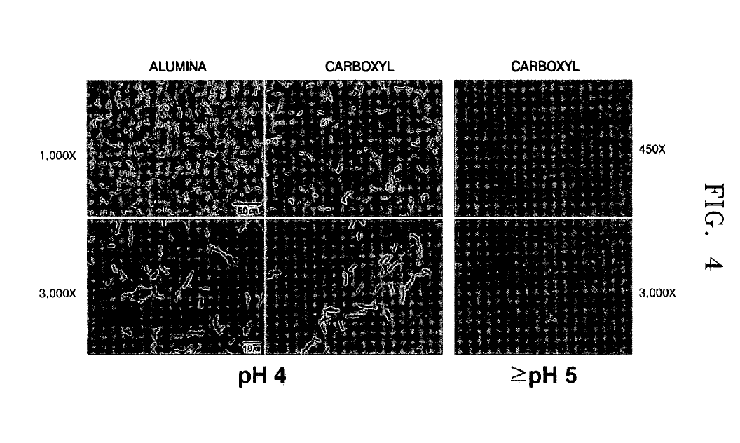

FIG. 4 is microscopic images showing the binding ability of Bacillus subtilis cells with respect to pH and the type of substrate. Referring to FIG. 4, cells satisfactorily bind to the alumina substrate and the carboxyl substrate at pH 4, while cells hardly bind to both of the alumina substrate (not shown) and the carboxyl substrate at pH 5 or greater.

### EXAMPLE 4

### Binding Ability of E. coli Cells with Respect to pH and Type of Substrate

To investigate the binding ability of cells with respect to pH and the type of substrate, E. coli cells and 0.1 M phosphate buffers of pH 4˜10 were used. The alumina substrate of the present invention and a carboxyl substrate (manufactured as described in Example 3) were used. The separation and staining procedures of cells were carried out in the same manner in Example 2, except that the binding time was 10 minutes instead of 30 minutes.

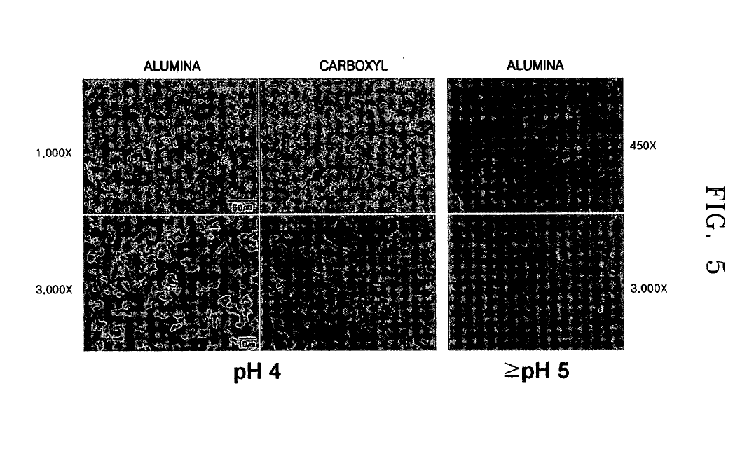

FIG. 5 is microscopic images showing the binding ability of E. coli cells with respect to pH and the type of substrate. Referring to FIG. 5, cells satisfactorily bind to both of the alumina substrate and the carboxyl substrate at pH 4, while cells hardly bind to both of the alumina substrate and the carboxyl substrate (not shown) at pH 5 or greater.

Thus, it can be seen that the pH of a cell solution significantly affects the binding ability of cells to the alumina substrate.

### EXAMPLE 5

### Binding Ability of E. coli Cells with Respect to Binding Time

To investigate the binding ability of cells with respect to binding time, E. coli cells, a 0.1 M phosphate buffer of pH 4, and the alumina substrate of the present invention were used. The separation and staining procedures of cells were carried out in the same manner in Example 2, except that the binding time was 1, 3 and 5 minutes instead of 30 minutes.

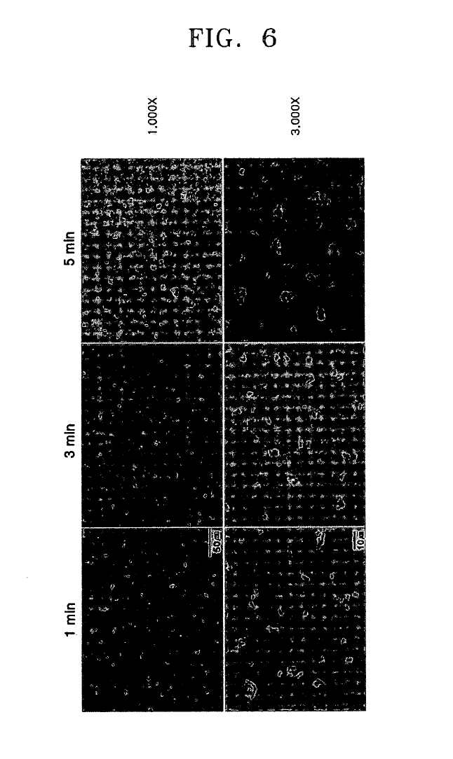

FIG. 6 is a microscopic image showing the binding ability of E. coli cells with respect to binding time. Referring to FIG. 6, there is no difference in the number of cells bound to the substrate with respect to various binding times within 5 minutes.

### EXAMPLE 6

### Binding Ability of Protein to Alumina Substrate of the Present Invention

To investigate whether proteins other than cells also bind to the alumina substrate of the present invention, Cy3-labeled IgG was used as a protein. 0.1 M phosphate buffers of pH 4, 7 and 10, and a carboxyl substrate (manufactured as described in Example 3), an amine substrate (Corning) and the alumina substrate of the present invention were used. 60 μl of Cy3-labeled IgG (pI 6.5˜9) was allowed to bind to each substrate for 60 minutes. The fluorescence intensity of IgG bound to each substrate was measured using an Axon scanner.

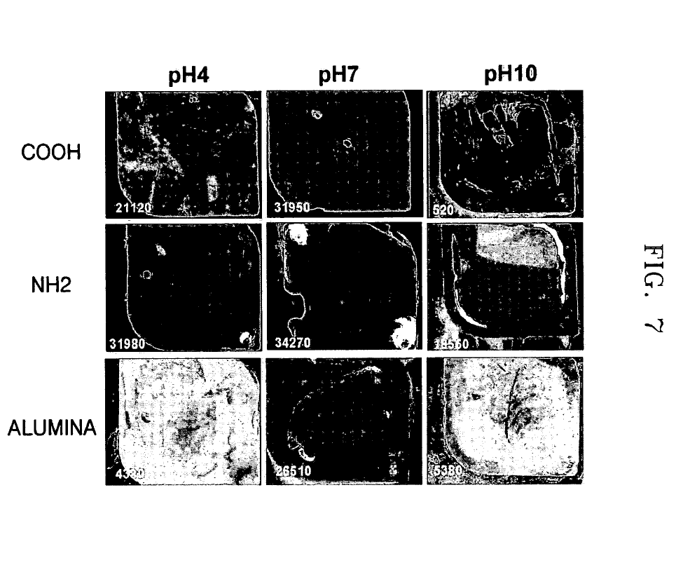

FIG. 7 shows fluorescence intensity of Cy3-labeled IgG bound to the carboxyl substrate, the amine substrate and the alumina substrate of the present invention with respect to pH. Referring to FIG. 7, at pH 10, the number of proteins bound to the alumina substrate is greater than that of proteins bound to the carboxyl substrate, while at pH 4 and 7, the number of proteins bound to the carboxyl substrate and the amine substrate is greater than that of proteins bound to the alumina substrate. In particular, at pH 4, the number of proteins bound to the carboxyl substrate and the amine substrate is much greater than that of proteins bound to the alumina substrate.

At pH 4 at which cells satisfactorily bind to the alumina substrate, the binding ability of proteins to the alumina substrate is significantly reduced, and thus the alumina substrate is suitable for separation of cells.

### EXAMPLE 7

### Binding Ability of Cells and Proteins to Alumina Substrate of the Present Invention

When cells and proteins were allowed to bind to the alumina substrate of the present invention at the same time, the influence of proteins on the binding ability of cells was investigated. E. coli cells were used and IgG were used as a protein. A 0.1 M phosphate buffer of pH 4 and the alumina substrate of the present invention were used. The separation and staining procedures of cells were carried out in the same manner as in Example 2, except that the binding time was 5 minutes instead of 30 minutes.

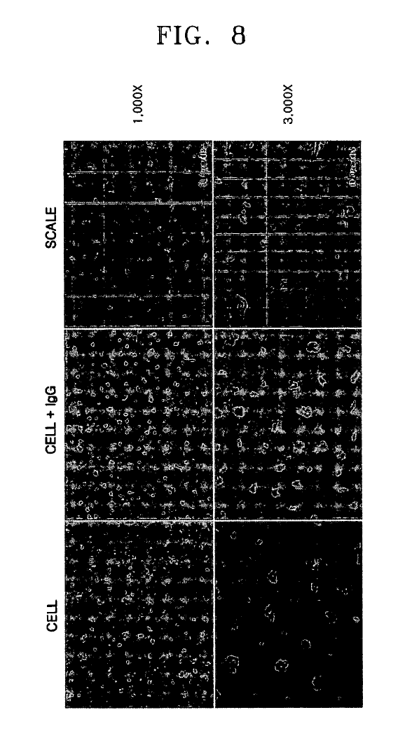

FIG. 8 is microscopic images showing the binding ability of E. coli cells to the alumina substrate with respect to the presence and absence of IgG. Referring to FIG. 8, it can be seen that proteins such as IgG do not affect the binding ability of a target cell to the alumina substrate.

When cells are lysed, the cell lysate contains proteins. The proteins do not bind to the alumina substrate and can be removed in a washing step. Thus, the influence of proteins on a subsequent PCR can be minimized.

### EXAMPLE 8

### DNA Binding Ability to Alumina Substrate of the Present Invention

To investigate the binding ability of DNA to the alumina substrate of the present invention with respect to the type of buffer, a 10×PCR buffer (Solgent; BSA 1 mg/mL), a 0.1 M phosphate buffer (pH 7.0) and a 0.1 N NaOH were used. 50 nM Cy3-labeled oligonucleotides (SEQ ID No: 1) as DNAs were allowed to bind to the alumina substrate at room temperature for 30 minutes. Then, the fluorescence intensity of DNAs bound to the alumina substrate was measured using an Axon scanner.

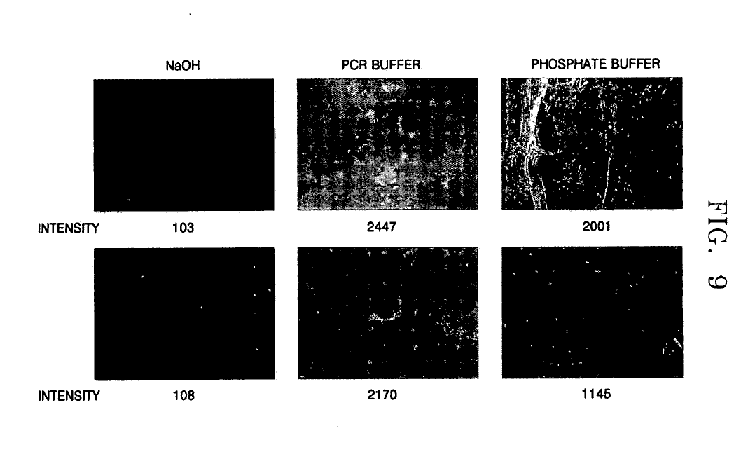

FIG. 9 shows the fluorescence intensity of DNA bound to the alumina substrate of the present invention with respect to the type of buffer. Referring to FIG. 9, unlike the Xtrana patent (U.S. Pat. No. 6,291,166), in the NaOH solution, DNAs hardly bind to the alumina substrate, while in 10× PCR buffer (Solgent; BSA 1 mg/mL) and the 0.1 M phosphate buffer (pH 7.0), DNAs bind to the alumina substrate. When cells bound to the alumina substrate were lysed using electrolysis, OH⁻ ions were generated in the cell lysate. DNAs in the cell lysate should not bind to the alumina substrate since they are used in a subsequent PCR. It can be seen from the above results that since DNAs hardly bind to the alumina substrate of the present invention in a NaOH solution, the alumina substrate which binds cells but does not bind DNAs is suitable for the purpose of the present invention.

### EXAMPLE 9

### PCR Amplification Efficiency of DNA Separated by the Method of the Present Invention

E. coli cells separated by the method of the present invention were lysed using electrolysis, and then the PCR amplification efficiency was investigated from released DNA. E. coli cells containing hepatitis B virus (HBV) plasmid DNA were used. The cell binding time was 5 minutes and electrolysis was carried out for 2 minutes. After the electrolysis, 5 μl of the electrolysed solution was used, a reaction volume was 20 μl and HBV plasmid DNA released from E. coli cells was used as a template to perform a PCR.

The PCR was performed in a LightCycler instrument (Roche Diagnostics, Mannheim, Germany) using a forward primer (SEQ ID No: 2) and a reverse primer (SEQ ID No: 3) to amplify a core region of the HBV genome. A mastermix of a LightCycler reaction contained the following ingredients: 2 μl of LightCycler master (Fast start DNA master SYBR Green I; Roche Diagnostics), 3.2 μl of MgCl₂ (5 mM), 1.0 μl of forward-reverse primer mixture (1.0 μM), 4.0 μl of UNG (Uracil-N-Glycosylase, 0.2 unit) and 4.8 μl of H₂O. Two types of Taq DNA polymerase (Roche Hot-start Taq DNA polymerase and Solgent Taq DNA polymerase) were used to prepare the LightCycler master.

For Roche Hot-start Taq DNA polymerase, predenaturation was performed at 50° C. for 10 minutes (for elimination of contaminated PCR products by Uracil-N-Glycosylase) and at 95° C. for 10 minutes, and then 40 cycles (denaturation at 95° C. for 5 seconds, and annealing and extension at 62° C. for 15 seconds) were performed. For Solgent Taq DNA polymerase, predenaturation was performed at 50° C. for 10 minutes (for elimination of contaminated PCR products by Uracil-N-Glycosylase) and at 95° C. for 1 minutes, and then 40 cycles (denaturation at 95° C. for 5 seconds, and annealing and extension at 62° C. for 15 seconds) were performed.

The amplified DNA was analyzed in Agilent 2100 Bioanalyzer (Agilent Technologies, Palo Alto, Calif.) with a commercial available DNA 500 assay sizing reagent set.

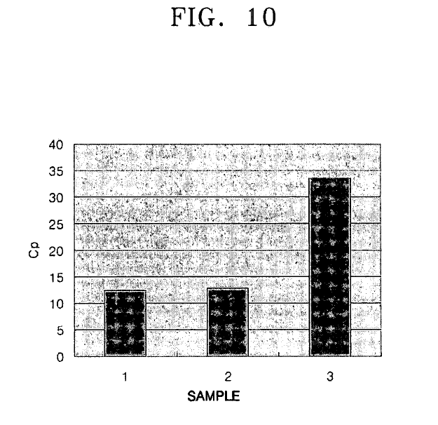

FIG. 10 illustrates the crossing point (Cp) of the respective samples in a PCR. Samples 1 and 2 are obtained from PCR using DNA separated by the method of the present invention and Sample 3 is obtained from PCR using distilled water as a negative control. Cp refers to the number of cycles at which the fluorescent signal is detected in a real-time PCR. That is, as the initial DNA concentration is higher, the fluorescent signal can be detected at a lower Cp. The Cp is also related to DNA purification. The higher DNA purity, the lower the Cp. Thus, it can be seen that as the Cp is lower, the DNA in the sample is a more specifically purified one.

As shown in FIG. 10, DNA separated by the method of the present invention has a significantly lower Cp than the negative control, which indicates that the number of the template DNA in the sample is great or the purity of the template DNA is very high.

Thus, using the separation method of nucleic acids according to the present invention, a PCR can be easily carried out. Therefore, the present invention is suitable for the implementation of LOC.

### EXAMPLE 10

### Binding Ability of Alumina Substrate of the Present Invention to Cells

To investigate the binding ability of the alumina substrate of the present invention to cells, E. coli cells, a 0.1 M phosphate buffer of pH 4 and the alumina substrate of the present invention were used. The number of initial cells was 1.00 E+09 cells/mL. The separation and staining procedures of cells were carried out in the same manner as in Example 2, except that the binding time was 5 minutes instead of 30 minutes.

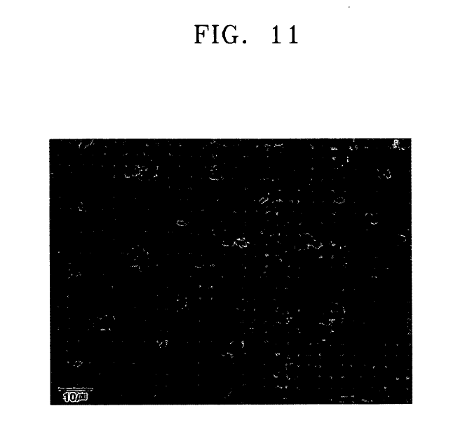

FIG. 11 is a microscopic image of E. coli cells bound to the alumina substrate of the present invention. Referring to FIG. 11, the number of cells adsorbed to the alumina substrate was 110 cells/8.00 E-05 cm².

As described above, the present invention can increase the separation efficiency of cells or viruses, improve detection limit for cells or viruses, and rapidly prepare a sample.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A method of separating cells or viruses using alumina, the method comprising:
contacting a solution containing cells or viruses with alumina; and
washing the alumina having cells or viruses bound thereto.

2. A method of separating nucleic acids from cells or viruses using alumina, the method comprising:
contacting a solution containing cells or viruses with alumina;
washing the alumina having cells or viruses bound thereto; and
lysing the cells or viruses bound to the alumina.

3. The method of claim 1, wherein the alumina is in the form of particle, membrane or substrate.

4. The method of claim 3, wherein the alumina is prepared from aluminium ethoxide or aluminium chloride.

5. The method of claim 4, wherein the alumina is prepared by sintering aluminium ethoxide or aluminium chloride at a temperature of 80-200° C.

6. The method of claim 1, wherein the cells or viruses contact the alumina at pH 3-4.5.

7. The method of claim 2, wherein the lysis of cells or viruses is carried out by electrolysis.

8. The method of claim 2, further comprising directly performing a nucleic acid amplification using the separated nucleic acids.

9. The method of claim 8, wherein the nucleic acid amplification is carried out by a PCR.

10. The method of claim 2, wherein the alumina is in the form of particle, membrane or substrate.

11. The method of claim 10, wherein the alumina is prepared from aluminium ethoxide or aluminium chloride.

12. The method of claim 11, wherein the alumina is prepared by sintering aluminium ethoxide or aluminium chloride at a temperature of 80-200° C.

13. The method of claim 2, wherein the cells or viruses contact the alumina at pH 3-4.5.


SEQUENCE LISTING (3 sequences)

SEQ ID NO: 1 - DNA, 50 bp, Artificial Sequence
  oligonucleotides
aggccagtggtatcgaccacctcccgctgtgggatgttgtgctgctgcac

SEQ ID NO: 2 - DNA, 20 bp, Artificial Sequence
  forward primer
agtgtggattcggcactcct

SEQ ID NO: 3 - DNA, 23 bp, Artificial Sequence
  reverse primer
gagttcttcttctaggggacctg